SEQUENCE LISTING (6 sequences)

SEQ ID NO: 1 - DNA, 26 bp, Artificial
  Synthetic AS1141
ggtggtggtggttgtggtggtggtgg

SEQ ID NO: 2 - DNA, 8 bp, Artificial
  Synthetic ISIS3052
ttggggtt

SEQ ID NO: 3 - DNA, 18 bp, Artificial
  Synthetic T30177
gtgggtgggtgggtgggt

SEQ ID NO: 4 - DNA, 16 bp, Artificial
  Synthetic T30923
gggtgggtgggtgggt

SEQ ID NO: 5 - DNA, 16 bp, Artificial
  Synthetic 93del
ggggtgggaggagggt

SEQ ID NO: 6 - DNA, 6 bp, Artificial
  Synthetic Hotoda′s
tgggag

(19) United States
(12) Patent Application Publication
(10) Pub. No.: US 2018/0036427 A1
(43) Pub. Date: Feb. 8, 2018

(54) METHODS AND PHARMACEUTICAL COMPOSITIONS FOR THE TREATMENT OF HIV INFECTION

(71) Applicants: INSERM (INSTITUT NATIONAL DE LA SANTÉ ETDE LA RECH MÉDICALE), Paris (FR); UNIVERSITÉ DE BORDEAUX, Bordeaux (FR); CVENTRE NATIONAL DE LA RECHERCHE SCIENTIFIQUE (CNRS), Paris (FR)

(72) Inventors: Samir AMRANE, Pessac (FR); Jean-Louis MERGNY, Pessac (FR); Marie-Aline ANDREOLA, Bordeaux (FR)

(21) Appl. No.: 15/555,121

(22) PCT Filed: Mar. 3, 2016
(86) PCT No.: PCT/EP2016/054499

§ 371 Date: Sep. 1, 2017

(30) Foreign Application Priority Data
Mar. 4, 2015 (EP) 15305329.3

(51) Int. Cl. A61K 47/69; A61K 31/713; A61K 45/06; C12N 15/115

(52) U.S. Cl. CPC A61K 47/6923; C12N 15/115; A61K 31/713; A61K 45/06; C12N 2310/16; C12N 2320/32; C12N 2320/31

(57) ABSTRACT

The present invention relates to methods and pharmaceutical compositions for the treatment of HIV infection. In particular, the present invention relates to a method of treating HIV infection in a subject in need thereof comprising administering the subject with a therapeutically effective amount of the oligonucleotide comprising the sequence as set forth in SEQ ID NO:

### FIELD OF THE INVENTION

The present invention relates to methods and pharmaceutical compositions for the treatment of HIV infection.

### BACKGROUND OF THE INVENTION

Human immunodeficiency virus (HIV-1) is a retrovirus responsible of a global pandemic inducing a deficiency of the immune system causing AIDS. HIV-1 retrovirus infects cells that carry CD4 and one of the chemokine receptors CCR5 or CXCR4. After infection, the two HIV-1 single-stranded RNAs are reverse transcribed by the viral reverse transcriptase into double-stranded DNA. The viral DNA is then integrated into the genome of the infected cell. The host cell machinery transcribes the viral genes, new viral proteins are synthesized, and new viruses are finally assembled. At the end of the 1990, the setting of an antiviral therapy targeting different enzymes of the viral cycle was a tremendous step forward in the battle against AIDS. However this treatment did not succeed into the definitive eradication of the virus and due to some mutations in the genome of the virus, resistance against these molecules can occur. Indeed, according to UNAIDS, 34 million people are currently infected by HIV-1. The discovery of new anti-viral strategies is still an important issue.

Nucleolin is a ubiquitous nucleolar phosphoprotein involved in fundamental aspects of transcription regulation, cell proliferation and growth. Nucleolin has also been described as a shuttling molecule between nucleus, cytosol and the cell surface. Studies have demonstrated that surface nucleolin may serve as a receptor for various extracellular ligands, for instance those implicated in cell proliferation, differentiation, adhesion, mitogenesis and angiogenesis. Nisole et al. (1999), US20040002457A1, and US20020076693A1 disclose that nucleolin is involved in binding of HIV virus to host cells. Anti-nucleolin aptamer AS1411 (also known as AGRO100) is a 26-base guanine-rich oligodeoxynucleotide aptamer with potential apoptotic induction activity but its role for inhibiting replication of HIV has never been investigated.

### SUMMARY OF THE INVENTION

The present invention relates to methods and pharmaceutical compositions for the treatment of HIV infection. In particular, the present invention is defined by the claims.

### DETAILED DESCRIPTION OF THE INVENTION

The present invention relates to a method of treating HIV infection in a subject in need thereof comprising administering the subject with a therapeutically effective amount of the oligonucleotide comprising the sequence as set forth in SEQ ID NO:1.

As used herein, the term “HIV” or “human immunodeficiency virus” refers to HIV-1, HIV-2, and any other strains of the virus which contribute to the development of AIDS or AIDS-related complex (ARC).

As used herein, the term “HIV infection” refers to any of the spectrum of conditions associated with HIV infection, ranging from asymptomatic seropositivity, through AIDS-related complex (ARC), to acquired immunodeficiency syndrome (AIDS).

As used herein, the term “treatment” or “treat” refers to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

The present invention contemplates the use of a guanosine-rich oligonucleotide called AS1411. AS1411 (as described in WO2009098464) has the sequence 5′-GGTGGTGGTGGTTGTGGTGGTGGTGG-3′ (SEQ ID NO: 1) and is also known as GRO26B and AGRO100. AS1411 is a 26-mer DNA aptamer, it has unmodified phosphodiester linkages and forms a G-quadruplex structure (Dapic, V. et al. 2003) that is resistant to degradation by serum enzymes (Dapic, V. et al. 2002). The structure was recently resolved (Chung W J, Heddi B, Schmitt E, Lim K W, Mechulam Y, Phan A T. Structure of a left-handed DNA G-quadruplex. Proc Natl Acad Sci USA. 2015 Feb. 18. pii: 201418718.).

For use in the instant invention, the oligonucleotide of the present invention is synthesized de novo using any of a number of procedures well known in the art. Chemical synthesis can be performed by a variety of automated nucleic acid synthesizers available in the market. These nucleic acids may be referred to as synthetic nucleic acids. Alternatively, the oligonucleotide of the present invention can be produced on a large scale in plasmids. The oligonucleotide of the present invention can be prepared from existing nucleic acid sequences using known techniques, such as those employing restriction enzymes, exonucleases or endonucleases.

In another embodiment, the oligonucleotide of the present invention is conjugated to nanoparticles to form nanoparticle-oligonucleotide conjugates. In some embodiments, the nanoparticles are a metal particles such as gold, silver, copper and platinum such as described in WO2005113817, Dam et al., 2015 and Malik et al., 2015.

By a “therapeutically effective amount” is meant a sufficient amount of the oligonucleotide of the present invention to treat the HIV infection at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The oligonucleotide of the present invention can be administered by known routes of administration including intravenous administration, intramuscular, intraperitoneal, intracerebrospinal, subcutaneous, intra-articular, intrasynovial, intrathecal, oral, topical, or inhalation routes. Effective dosages and schedules for administering antagonists or agonists are determined empirically according to guidelines generally recognized by those of skill in the art. Single or multiple dosages may be employed.

As noted above, the oligonucleotide of the present invention can be incorporated into pharmaceutical compositions suitable for administration into an animal such as a mammal. Methods for formulating such compositions are generally well known. Guidance is available for example from Remington: THE SCIENCE AND PRACTICE OF PHARMACY, 19th Edition, Gennaro (ed.) 1995, Mack Publishing Company, Easton, Pa. Such compositions typically comprise at least one oligonucleotide of the present invention and a pharmaceutically acceptable carrier. The term “pharmaceutically acceptable carrier” refers to any and all coatings, excipients, solvents, dispersion media, absorption delaying agents, and the like, compatible with pharmaceutical administration. Such carriers also include for example sodium chloride, colloidal silica, talc, various polymeric carriers including polyvinyl pyrrolidone, cellulose-based compounds such as carboxymethylcellulose or methylcellulose, polyvinylpyrrolidone, polyacrylates, and polyethylene glycol. Dosage forms include, for example, oral or sublingual tablets, pellets, micro- and nano-capsules, liposomes, inhalation forms, nasal sprays, and sustained-release preparations. Solutions or suspensions used for administering nucleic acids of the present invention can include one or more of the following components: a sterile diluent such as water for injection, saline solution; fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose. In some embodiments, a pharmaceutical composition can be delivered via slow release formulation or matrix comprising nucleic acids of the present invention or DNA constructs suitable for expression of nucleic acids of the present invention in or around a site within the body.

In some embodiments, the oligonucleotide of the present invention of the invention may be formulated into pharmaceutical compositions that can be used to apply microbicides to effectively prevent transmission of HIV through mucosae, more particularly to prevent the sexual or vaginal transmission of HIV. Thus, the compositions are in forms adapted to be applied to the site where sexual intercourse or related intimate contact takes place, such as the genitals, vagina, vulva, cervix, rectum, mouth, hands, lower abdomen, upper thighs, especially the vagina, vulva, cervix, and ano-rectal mucosae. As appropriate topical compositions there may be cited for example gels, jellies, creams, pastes, emulsions, dispersions, ointments, films, sponges, foams, aerosols, powders, intravaginal rings or other intravaginal drug delivery systems, cervical caps, implants, patches, suppositories or pessaries for rectal, or vaginal application, vaginal or rectal or buccal tablets, mouthwashes. The present topical formulations such as the gel formulations described herein could, for example, be applied into the vagina by hand, suppositories, or conventional tampon or syringe techniques. The method of administering or delivering the gel into the vagina is not critical so long as an effective amount of the gel is delivered into the vagina. The present topical formulations such as the gel formulations described herein may also be used for protection during anal intercourse and can be applied using similar techniques. For vaginal heterosexual intercourse, the present topical formulations such as the gel formulations described herein may be applied into the vagina prior to intercourse. For anal intercourse (heterosexual or homosexual), the present topical formulations such as the gel formulations described herein may be inserted into the rectum prior to intercourse. For either vaginal or anal intercourse, the present topical formulations such as the gel formulations described herein may also act as a lubricant. For added protection it is generally preferred that the present topical formulations such as the gel formulations described herein be applied before intercourse or other sexual activity and that, if appropriate, a condom be used. For even further protection, the present topical formulations such as the gel formulations described herein can be applied as soon as possible after completion of the sexual activity. Although application only after the sexual activity is less recommended, it would still be desirable afterwards if the application was not performed prior to the sexual activity for any reason (e.g., in cases of rape).

In some embodiments, the oligonucleotide of the present invention may be used in all the suitable formulations, alone or in combination with other active ingredients, such as antivirals, antibiotics, immunomodulators or vaccines. They may also be used alone or in combination with other prophylactic agents for the prevention of viral infections. Thus, the oligonucleotide of the present invention may be combined with pharmaceutically acceptable adjuvants conventionally employed in vaccines and administered in prophylactically effective amounts to protect individuals over an extended period of time against HIV-1 infection. Antiviral compounds which may be used in combination with the oligonucleotide of the present invention may be known antiretroviral compounds such as pentamidine, thymopentin, castanospermine, dextran (dextran sulfate), foscarnet-sodium (trisodium phosphono formate); nucleoside reverse transcriptase inhibitors, e.g. zidovudine (3′-azido-3′-deoxythymidine, AZT), didanosine (2′,3′-dideoxyinosine; ddI), zalcitabine (dideoxycytidine, ddC) or lamivudine (2′-3′-dideoxy-3′-thiacytidine, 3TC), stavudine (2′,3′-didehydro-3′-deoxythymidine, d4T), abacavir and the like; non-nucleoside reverse transcriptase inhibitors such as nevirapine (11-cyclopropyl-5,11-dihydro-4-methyl-6H-dipyrido-[3,2-b:2′,3′-e] [1,4]diazepin-6-one), efavirenz, delavirdine, and the like; phosphonate reverse transcriptase inhibitors, e.g. tenofovir and the like; compounds of the TIBO (tetrahydro-imidazo[4,5,1-jk][1,4]-benzodiazepine-2(1H)-one and thione)-type e.g. (S)-8-chloro-4,5,6,7-tetrahydro-5-methyl-6-(3-methyl-2-butenyl)imidazo-[4,5,1-jk][1,4]benzo-diazepine-2(1H)-thione; compounds of the [alpha]-APA ([alpha]-anilino phenyl acetamide) type e.g. [alpha]-[(2-nitrophenyl)amino]-2,6-dichlorobenzene-acetamide and the like; inhibitors of trans-activating proteins, such as TAT-inhibitors, e.g. RO-5-3335, or REV inhibitors, and the like; protease inhibitors e.g. indinavir, ritonavir, saquinavir, lopinavir (ABT-378), nelfinavir, amprenavir, TMC-126, BMS-232632, VX-175 and the like; fusion inhibitors, e.g. T-20, T-1249 and the like; CXCR4 receptor antagonists, e.g. AMD-3100 and the like; inhibitors of the viral integrase; ribonucleotide reductase inhibitors, e.g. hydroxyurea and the like. Combinations may as well exert a synergistic effect in inhibiting HIV replication when components of the combination act on different or same sites of HIV replication, preferably on different sites. The use of such combinations may reduce the dosage of a given conventional antiretroviral agent which would be required for a desired prophylactic effect as compared to when that agent is administered as a single active ingredient. These combinations reduce potential of resistance to single agent, while minimizing any associated toxicity. These combinations may also increase the efficacy of the conventional agent without increasing the associated toxicity.

The invention will be further illustrated by the following figures and examples. However, these examples and figures should not be interpreted in any way as limiting the scope of the present invention.

### FIGURES

FIG. 1: A. Schematic representation of a tetrad composed of 4 guanine nucleosides. B. Example of a G-quadruplex structure based on the the stacking of 3 tetrads C. Viral replication was monitored by β-galactosidase activity at 24 h post-infection. Values were normalized to 100% corresponding to β-galactosidase activity in the absence of oligonucleotide. The dose effects are performed in the presence of AS1411 (losange), T30923 (cross), T30177 (circles), ISIS3052 (squares).

FIG. 2:

A. Tetrad arrangement of guanosine residues. The monovalent cation that stabilizes this tetrad is represented by a plus sign. B. Representative inhibition curves obtained in the cellular-based assay. C. Antiviral activity of AS1411 and other G-quadruplex forming inhibitors. Phosphorothioate positions are shown with a star. EC50 and standard deviations (SD) are derived from 4 independent experiments unless reported from the literature (a). Data reported in italic are from experimental settings that are different from those used in the present study.

### EXAMPLES

### Example 1

Material and Methods:

Preparation of the Oligonucleotides:

Oligonucleotides were purchased from Eurogentec (Seraing, Belgium) with “Reverse-Phase Cartridge Gold purification”. Concentrations were determined by ultraviolet (UV) absorption using the extinction coefficients provided by the manufacturer. All oligonucleotides were dissolved in 20 mM potassium phosphate buffer pH7 containing 70 mM KCl.

Cell Lines and Viruses:

HeLa P4 cells encoding a Tat-inducible β-galactosidase were maintained in DMEM medium (Invitrogen) supplemented with 10% inactivated FCS, 1 mg/ml geneticin (G418, Gibco-BRL), gentamycin. MT4 and H9_{Lai} cells were grown in RPMI 1640 glutamax medium (Invitrogen) supplemented with 10% inactivated FCS. HIV-1 viruses were obtained after 48 h co-culture of MT4 cells (0.5×106/ml) and H9Laï cells (1×106/ml), chronically infected by HIV-1Lai isolate, in RPMI 1640 glutamax medium supplemented with 10% inactivated FCS, at 37° C. under humidified atmosphere and 5% CO2. The culture was then centrifuged and the supernatant was clarified by filtration on a 0.45 μm membrane before freezing at −80° C.

Viral Infectivity Test:

The oligonucleotides were preincubated in a 100 mM potassium solution to favour G4 formation. When added, they are incubated in presence of the HelaP4 cells 20 minutes before infection. The infectivity was assayed on HeLa P4 cells expressing CD4 receptor and the β-galactosidase gene under the control of the HIV-1 LTR. HeLa P4 were plated using 200 μl of DMEM medium supplemented with 10% inactivated FCS in 96-multi-well plates at 10 000 cells per well. After overnight incubation at 37° C., under humidified atmosphere and 5% CO₂, the supernatant was discarded and 200 μl of viral preparation were added in serial dilutions. After 24 h of infection, the supernatant was discarded and the wells were washed 3 times with 200 μl of PBS. Each well was refilled with 200 μl of a reaction buffer containing 50 mM Tris-HCl pH 8.5, 100 mM β-mercaptoethanol, 0.05% Triton X-100 and 5 mM 4-methylumbelliferyl-B-D-galactopyranoside (4-MUG) (Sigma). After 24 h, the reaction was measured in a fluorescence microplate reader (Cytofluor II, Applied Biosystems) at 360/460 nm Ex/Em.

Results:

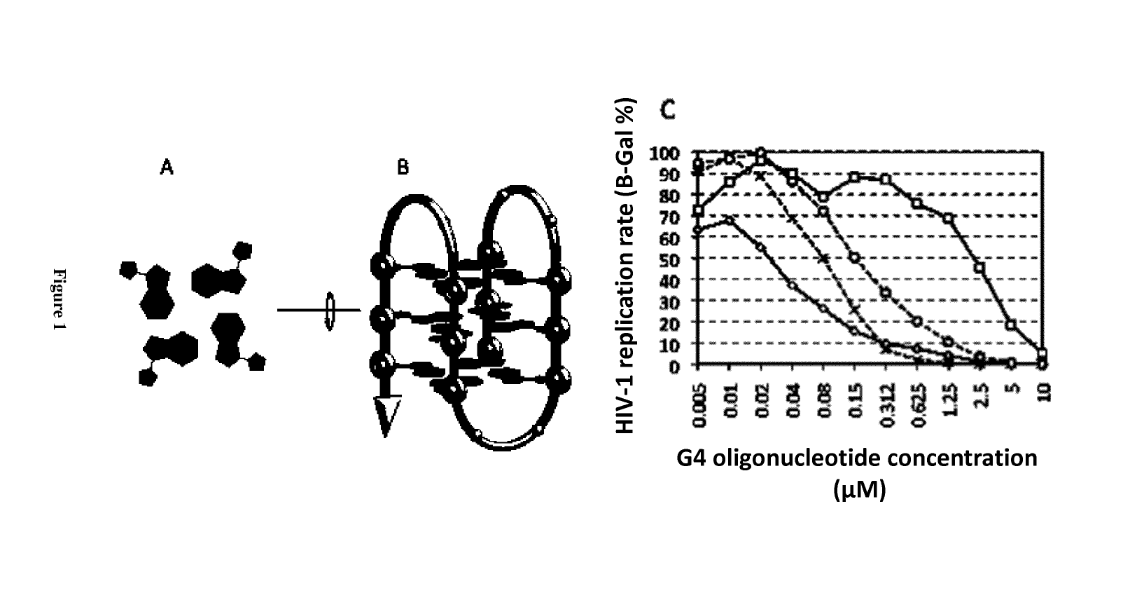

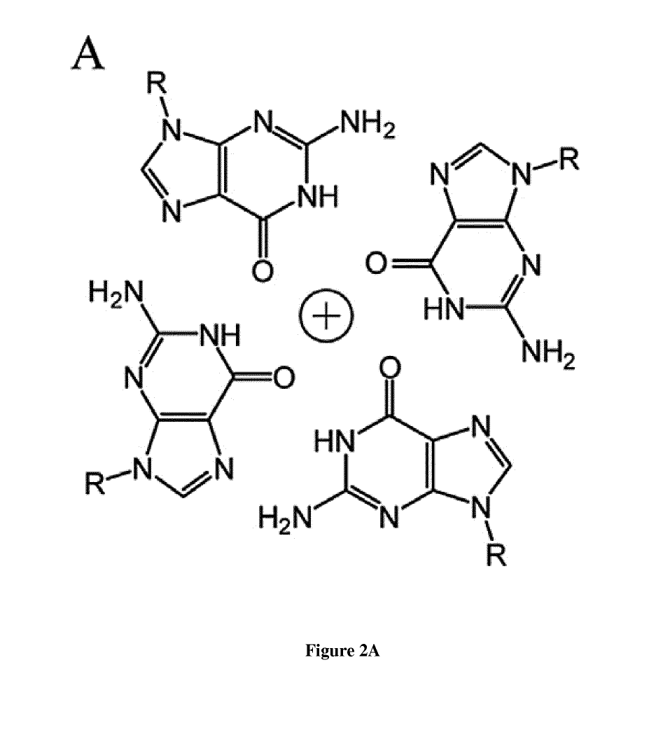

The anti-HIV properties reported in the literature for diverse G4 forming aptamers (93del, T30177 . . . ) prompted us to test if AS1411 anti-cancer G4 aptamer is also able to inhibit HIV-1 infectivity. HeLa P4 cells were infected by viral supernatant HIV-1 as described in the Methods section. To assess the inhibitory effect of the aptamers, HeLa P4 cells were infected with HIV-1 in the presence of increasing concentrations of ISIS3052, T30177, T30923 and AS1411 ranging from 0.005 μM till 10 μM. All the G4s were able to inhibit HIV-1 infectivity with IC₅₀ ranging from 25 to 2500 nM (FIG. 1). Interestingly, AS1411 was the strongest HIV-1 inhibitor with an IC₅₀ of 25 nM.

TABLE 1

| | | HIV | HIV |
|---|---|---|---|
| | | Inhibition^{a} | Inhibition^{b} |
| Aptamer | Sequence | IC₅₀ (nM) | IC₅₀ (nM) |
| ISIS3052 | TTGGGGTT | 2500 | >1000 |
| | (SEQ ID NO: 2) | | |
| T30177 | GTGGGTGGGTGGGTGGGT | 150 | ~100 |
| | (SEQ ID NO: 3) | | |
| T30923 | GGGTGGGTGGGTGGGT | 80 | ~100 |
| | (SEQ ID NO: 4) | | |
| 93del | GGGGTGGGAGGAGGGT | 20-30^{c} | — |
| | (SEQ ID NO: 5) | | |
| Hotoda's | TGGGAG | — | >1000 |
| | (SEQ ID NO: 6) | | |
| AS1411 | GGTGGTGGTGGTTGTGGT | 25 | Not |
| | GGTGGTGG | | determined |
| | (SEQ ID NO: 1) | | before- |

^{a}IC₅₀ inhibition were determined by us using the HIV-1 infectivity test described in methods
^{b}IC₅₀ inhibition data from the literature using different HIV-1 infectivity test
^{c}data not shown

### Example 2

Material and Methods:

Oligonucleotides

Oligonucleotides were purchased from Eurogentec (Seraing, Belgium) with “Reverse-Phase Cartridge Gold purification” and dissolved in 20 mM potassium phosphate buffer pH 7.0 containing 70 mM KCl.

Antiviral Activity

Infectivity of replicative HIV-1 particles was monitored as previously reported [30]. Briefly, HeLaP4 cells are reporter cells that contain a LacZ gene integrated in their genome, the expression of which is under the control of the viral LTR promoter. Antiviral activity of molecules was monitored 24 h post-infection. Fluorescence associated with the reaction product was monitored using a Cytofluor-II plate reader (Applied Biosystems, Foster City, Calif.) with excitation/emission filters at 360/460 nm. Data analysis (non-linear regression, IC50 determination and standard deviation) was performed using Prism 5.0c (GraphPad).

Results:

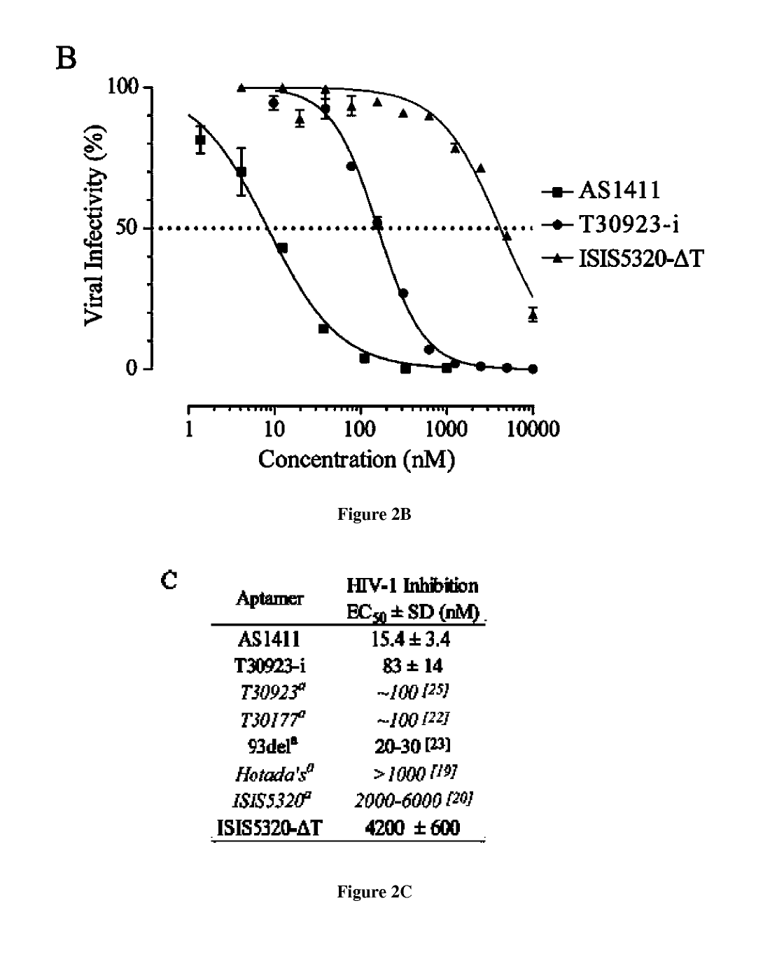

In the present invention, the inventors assessed whether AS1411 was able to inhibit HIV-1 replication in a cellular context. Interestingly, AS1411 exhibited anti-HIV-1 activity at low nanomolar concentrations with an EC50 of only 15.4±3.4 nM (FIG. 2B-C). T30923-i is a close derivative of T30923 with a single guanosine to inosine substitution (FIG. 2C) forming a G-quadruplex as shown by NMR (Do et al., 2011). T30923-I was also a potent antiviral with an EC50 of 83±14 nM (FIG. 2B-C), which is consistent with previous results obtained with T30923 (Ojwang et al., 1995; Jing et al., 20001; Rando et al., 1995). Although zintevir was specifically developed as antiviral agent and was evaluated in clinical trials, AS1411 was 5- to 6-times more efficient at inhibiting viral replication than any of zintevir's derivatives tested so far (FIG. 2C). In parallel, we also tested ISIS5320-DT that derives from ISIS5320 and forms a well-defined G-quadruplex structure (Caceres et al., 2004). In the same cellular assay, ISIS5320-DT failed at inhibiting HIV-1 replication at nanomolar concentrations (EC50 of 4.2±0.6 mM, FIG. 2B-C), which was similar to the antiviral activity of the parent molecule ISIS5320 previously reported (Stoddart et al., 1998). Thus, AS1411 is the most potent antiviral molecule within the nucleic acids/G-quadruplex forming oligonucleotides family tested so far, including zintevir and andevir (FIG. 2C).

During clinical trials of AS1411 for its potential anticancer applications, it appeared that the molecule was safe but rapidly eliminated from the human body (Bates et al., 2009). This could well also be a limiting factor for its use as antiviral drug. However, we showed here that AS1411 was a potent antiviral at 1000-fold lower concentrations than the concentrations needed to obtain an effective anticancer activity. Thus, AS1411 could, in its present form, present great therapeutic value as an anti-HIV agent. Alternatively, recent studies showed that association of AS1411 with gold particles enhances its effectiveness in in vivo cancer models (Dam et al., 2015; Malik et al., 2015) with an enhanced bioavailability an no increase in toxicity. Therefore, AS1411 alone or conjugated to nanoparticles represents a serious candidate for anti-HIV applications.

### REFERENCES

Throughout this application, various references describe the state of the art to which this invention pertains. The disclosures of these references are hereby incorporated by reference into the present disclosure.
Dapić V, Bates P J, Trent J O, Rodger A, Thomas S D, Miller D M. Antiproliferative activity of G-quartet-forming oligonucleotides with backbone and sugar modifications. Biochemistry. 2002 Mar. 19; 41(11):3676-85.
Dapić V, Abdomerović V, Marrington R, Peberdy J, Rodger A, Trent J O, Bates P J. Biophysical and biological properties of quadruplex oligodeoxyribonucleotides. Nucleic Acids Res. 2003 Apr. 15; 31(8):2097-107.
Chung W J, Heddi B, Schmitt E, Lim K W, Mechulam Y, Phan A T. Structure of a left-handed DNA G-quadruplex. Proc Natl Acad Sci USA. 2015 Feb. 18.
N. Q. Do, K. W. Lim, M. H. Teo, B. Heddi, A. T. Phan, Stacking of G-quadruplexes: NMR structure of a G-rich oligonucleotide with potential anti-HIV and anticancer activity, Nucleic Acids Res. 39 (2011) 9448e9457.
J. O. Ojwang, R. W. Buckheit, Y. Pommier, A. Mazumder, K. De Vreese, J. A. Este, et al., T30177, an oligonucleotide stabilized by an intramolecular guanosine octet, is a potent inhibitor of laboratory strains and clinical isolates of human immunodeficiency virus type 1, Antimicrob. Agents Chemother. 39 (1995) 2426e2435.
N. Jing, C. Marchand, Y. Guan, J. Liu, L. Pallansch, C. Lackman-Smith, et al., Structure-activity of inhibition of HIV-1 integrase and virus replication by Gquartet oligonucleotides, DNA Cell Biol. 20 (2001) 499e508.
R. F. Rando, J. Ojwang, A. Elbaggari, G. R. Reyes, R. Tinder, M. S. McGrath, et al., Suppression of human immunodeficiency virus type 1 activity in vitro by oligonucleotides which form intramolecular tetrads, J. Biol. Chem. 270 (1995) 1754e1760.
C. Caceres, G. Wright, C. Gouyette, G. Parkinson, J. A. Subirana, A thymine tetrad in quadruplexes stabilized with Tlρ/Naρ ions, Nucleic Acids Res. 32 (2004) 1097e1102.
C. A. Stoddart, L. Rabin, M. Hincenbergs, M. Moreno, V. Linquist-Stepps, J. M. Leeds, et al., Inhibition of human immunodeficiency virus type 1 infection in SCID-hu Thy/Liv mice by the G-quartet-forming oligonucleotide, ISIS 5320, Antimicrob. Agents Chemother. 42 (1998) 2113e2115.
P. J. Bates, D. A. Laber, D. M. Miller, S. D. Thomas, J. O. Trent, Discovery and development of the G-rich oligonucleotide AS1411 as a novel treatment for cancer, Exp. Mol. Pathol. 86 (2009) 151e164.
D. H. Dam, K. S. Culver, I. Kandela, R. C. Lee, K. Chandra, H. Lee, et al., Biodistribution and in vivo toxicity of aptamer-loaded gold nanostars, Nanomedicine 11 (2015) 671e679.
M. T. Malik, M. G. O'Toole, L. K. Casson, S. D. Thomas, G. T. Bardi, E. M. Reyes-Reyes, et al., AS1411-conjugated gold nanospheres and their potential for breast cancer therapy, Oncotarget 6 (2015) 22270e22281.

1. A method of treating HIV infection in a subject in need thereof comprising administering the subject with a therapeutically effective amount of the oligonucleotide comprising the sequence as set forth in SEQ ID NO:1.

2. The method of claim 1 wherein said oligonucleotide is conjugated to nanoparticles.

3. The method of claim 2 wherein said nanoparticles are gold nanoparticles.

4. The method of claim 1 wherein the oligonucleotide is used in combination with at least one antiretroviral compound selected from the group consisting of pentamidine, thymopentin, castanospermine, dextran, foscarnet-sodium; nucleoside reverse transcriptase inhibitors, non-nucleoside reverse transcriptase inhibitors compounds of the TIBO; compounds of the [alpha]-APA ([alpha]-anilino phenyl acetamide); inhibitors of trans-activating proteins, such as TAT-inhibitors, protease inhibitors fusion inhibitors, inhibitors of the viral integrase; and ribonucleotide reductase inhibitors.
